(19) United States
(12) Patent Application Publication
(10) Pub. No.: US 2019/0000868 A1
(43) Pub. Date: Jan. 3, 2019

(54) COMPOSITIONS COMPRISING 4'-O-GLUCURONIDE EPICATECHIN AND METHODS OF MAKING AND USING SUCH COMPOSITIONS

(71) Applicant: NESTEC S.A., Vevey (CH)

(72) Inventors: Lucas Actis Goretta, Singapore (SG); Amaury Patin, Lausanne 26 (CH); Stephanie Michlig Gonzalez, Le Mont-sur-Lausanne (CH); Susana Camacho, Lutry (CH); Coline Legrand, Lausanne (CH); Johannes Le Coutre, Pully (CH); Euridice Castaneda Gutierrez, Belmont-sur-Lausanne (CH); Irma Silva Zolezzi, Carrouge (CH)

(73) Assignee: NESTEC S.A., Vevey (CH)

(21) Appl. No.: 16/062,907

(22) PCT Filed: Dec. 15, 2016
(86) PCT No.: PCT/EP2016/081230

§ 371 Date: Jun. 15, 2018

Related U.S. Application Data
(60) Provisional application No. 62/268,147, filed on Dec. 16, 2015.
(60) Provisional application No. 62/286,688, filed on Jan. 25, 2016.

(51) Int. Cl. A61K 31/7048; A61P 9/12

(52) U.S. Cl. CPC A61K 31/7048; A61P 9/12

(57) ABSTRACT

Compositions can comprise the flavanol metabolite 4′-O-glucuronide epicatechin (4GEC). In some embodiments, the composition can be used in a method for blood vessel dilation and/or increased delivery of blood flow to tissues in the body, for example by administering the composition to an individual having or at risk of high blood pressure or a cardiovascular disease. In some embodiments, the compositions are used for weight maintenance or weight loss. The compositions are preferably administered orally as a food product in which the 4′-Oglucuronide is present in a concentration of at least 0.01 mg/g of the food product.

### BACKGROUND

The present disclosure generally relates to compositions comprising a flavanol metabolite. More specifically, the present disclosure relates to compositions comprising the flavanol metabolite 4′-O-glucuronide epicatechin and further relates to methods comprising administering such compositions and methods of making such compositions.

A condition adversely affecting some individuals is high blood pressure. Blood pressure is the force of blood pushing against the walls of the arteries as the heart pumps out blood. High blood pressure is a serious condition that is associated with a higher risk of cardiovascular diseases and can lead to, for example, coronary heart disease, heart failure, stroke, kidney failure, and other health problems.

Hypertension is a condition caused by a sustained high blood pressure. Hypertension is a cardiac chronic medical condition in which the systemic arterial blood pressure is outside a normal range. Hypertension generally refers to a condition where a systolic blood pressure is 140 mmHg or higher or a diastolic blood pressure is 90 mmHg or higher. Hypertension is classified as either primary or secondary. About 90-95% of hypertension cases are primary hypertension, which refers to high blood pressure for which no medical cause has been found. The remaining 5-10% of cases are secondary hypertension, which refers to high blood pressure caused by other conditions that affect the kidneys, arteries, heart, or endocrine system.

The incidence of hypertension is increasing all over the world. In addition, hypertension may cause fatal complications such as cerebral stroke, heart failure, and coronary artery diseases, even among minor or mild patients exhibiting no external symptoms.

Hypertension is commonly addressed with interventions that are not fully effective and thus could benefit from improved methods and compositions. High blood pressure and hypertension can also be problems during pregnancy.

### SUMMARY

Without wishing to be bound by theory, the present inventors believe that 4′-O-glucuronide epicatechin is effective to modulate nitric oxide levels to achieve blood vessel dilation and/or increased delivery of blood flow to tissues in the body. In turn, the blood vessel dilation and/or increased delivery of blood flow can reduce blood pressure, stimulate protein synthesis, improve blood circulation, improve blood brain circulation increase release of growth factors, enhance immune function, and/or improve one or more of insulin sensitivity, glucose tolerance, mood, memory or cognition.

Accordingly, in a general embodiment, the present disclosure provides a method for blood vessel dilation and/or increased delivery of blood flow to tissues in the body. The method comprises administering a composition comprising 4′-O-glucuronide epicatechin to an individual.

In an embodiment, the composition is orally administered to the individual as a food product in which the 4′-O-glucuronide epicatechin is present in a concentration of at least 0.01 mg/g of the food product.

In an embodiment, the composition is administered to the individual at least once a day for at least one week.

In an embodiment, the 4′-O-glucuronide epicatechin is chemically synthesized.

In an embodiment, the individual has or is at risk of high blood pressure.

In an embodiment, the individual has or is at risk of a cardiovascular disease. In an embodiment the composition is administered to prevent preeclampsia and/or IUGR.

In an embodiment, the composition is administered to the individual in an amount that achieves a therapeutic effect selected from the group consisting of reduced blood pressure, stimulation of protein synthesis, improve blood circulation, improve blood brain circulation, increased release of growth factors, enhanced immune function, and combinations thereof, the method comprising administering to the individual a composition comprising 4′-O-glucuronide epicatechin.

In an embodiment, the composition is administered to the individual in an amount that improves a characteristic selected from the group consisting of insulin resistance, glucose tolerance and a combination thereof. The individual can be selected from the group consisting of an infant born preterm, an infant experiencing intrauterine growth restriction, a pregnant woman, a pregnant woman suffering from gestational diabetes or at risk of suffering from gestational diabetes mellitus, a human suffering from insulin resistance, a human suffering from impaired glucose tolerance, and a human suffering from type II diabetes.

In an embodiment, the composition is administered to the individual in an amount that improves one or more of cognitive performance, cognition, mood, or memory. The individual can have a condition selected from the group consisting of cognitive decline, cognitive aging, mild cognitive impairment, dementia, a mood disorder, memory loss, and combinations thereof. For example, the individual can be an elderly human having cognitive aging.

In an embodiment, the composition is injected into the individual.

In another embodiment, a method for weight maintenance or weight loss is provided. The method comprises administering to an individual a composition comprising 4′-O-glucuronide epicatechin. The composition can be orally administered to the individual as a food product in which the 4′-O-glucuronide epicatechin is present in a concentration of at least 0.01 mg/g of the food product and/or can be administered to the individual at least once a day for at least one week. In an embodiment, the 4′-O-glucuronide epicatechin is chemically synthesized. The composition can be injected into the individual.

In an embodiment, the composition further comprises an additional ingredient in an amount effective to promote weight maintenance or weight loss.

In an embodiment, the composition is administered to provide an amount of the 4′-O-glucuronide epicatechin that increases at least one characteristic selected from the group consisting of energy expenditure, sympathetic nervous system activity, and fat oxidation.

In an embodiment, the individual has or is at risk of obesity or overweight.

In an embodiment, the individual is participating in a weight loss program selected from the group consisting of a low-fat diet, a low-carbohydrate diet, a low-calorie diet, a very low-calorie diet, endurance training, strength training, and combinations thereof.

In another embodiment, the present disclosure provides a method for treating or preventing endothelium dysfunction comprising administering to an individual in need thereof or at risk thereof a composition comprising 4′-O-glucuronide epicatechin. The composition can be orally administered to the individual as a food product in which the 4′-O-glucuronide epicatechin is present in a concentration of at least 0.01 mg/g of the food product. It can be ingested in a special form to prevent degradation in the stomach. The composition can be administered to the individual at least once a day for at least one week.

An advantage of the present disclosure is to use a compound that is easily consumed.

Another advantage of the present disclosure is to provide tolerable side effects or no side effects while achieving blood vessel dilation and/or increased delivery of blood flow to tissues in the body.

Yet another advantage of the present disclosure is to use a compound that has increased acceptability, reduced pungency, and improved tolerance in the gastrointestinal tract relative to capsaicin.

Still another advantage of the present disclosure is to treat or prevent high blood pressure.

An additional advantage of the present disclosure is to treat or prevent cardiovascular diseases.

Additional features and advantages are described herein, and will be apparent from, the following Detailed Description and the Figures.

### BRIEF DESCRIPTION OF THE FIGURES

FIG. 1 shows the chemical structure of 4′-O-glucuronide epicatechin.

FIG. 2 shows the chemical structure of a first compound for the production of 4′-O-glucuronide epicatechin.

FIG. 3 shows the chemical structure of a second compound which can be coupled to the first compound shown in FIG. 2 for the production of 4′-O-glucuronide epicatechin.

FIG. 4 shows comparative experimental data for 4′-O-glucuronide epicatechin versus other metabolites on expression of p-eNOS and eNOS total in isolated aortic rat rings.

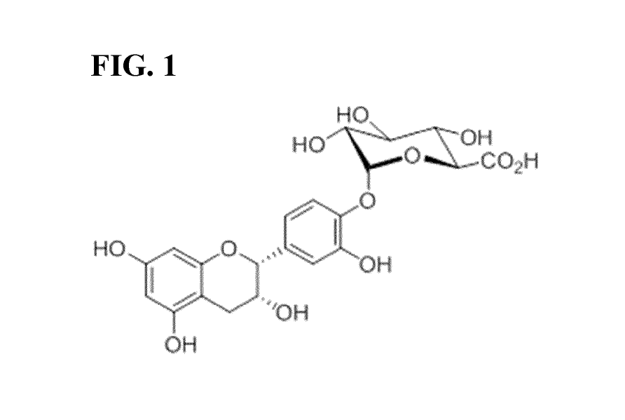

### DETAILED DESCRIPTION

All percentages expressed herein are by weight of the total weight of the composition unless expressed otherwise. When reference is made to the pH, values correspond to pH measured at 25° C. with standard equipment. As used in this disclosure and the appended claims, the singular forms “a,” “an” and “the” include plural referents unless the context clearly dictates otherwise. As used herein, “about” is understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. Moreover, all numerical ranges herein should be understood to include all integers, whole or fractions, within the range. The compositions disclosed herein may lack any element that is not specifically disclosed herein. Thus, a disclosure of an embodiment using the term “comprising” includes a disclosure of embodiments “consisting essentially of” and “consisting of” the components identified.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

For adults, “high blood pressure” is a systolic blood pressure of 140 mmHg or higher and/or a diastolic blood pressure of 90 mmHg or higher. High blood pressure includes both primary and secondary hypertension. Non-limiting examples of individuals “at risk” of high blood pressure include humans of age 60 or older, overweight or obese individuals, individuals who smoke at least once a day, humans who consume at least 2.4 g of sodium each day, humans who consume less than 4.7 g of potassium each day, individuals who perform aerobic exercise less than 3 days per week, men who consume more than 3 units of alcohol per day, women who consume more than 2 units of alcohol per day, individuals who have a mother or father with high blood pressure, and individuals having either (i) a systolic blood pressure from 120 to 139 mmHg and a diastolic blood pressure from 40 to 80 mmHg or (ii) a systolic blood pressure from 70 to 140 mmHg and a diastolic blood pressure from 80 to 89 mmHg.

Cardiovascular diseases are diseases associated with high blood pressure. Non-limiting examples of cardiovascular diseases include coronary heart disease, heart failure, peripheral arterial disease, hypertensive retinopathy, hypertensive encephalopathy, stroke, kidney failure, and combinations thereof. Non-limiting examples of individuals “at risk” of a cardiovascular disease include individuals with high blood pressure, individuals at risk of high blood pressure, and individuals with high blood cholesterol (e.g., total cholesterol of 240 mg/dL or greater and/or LDL (low-density lipoprotein) of 160 mg/dL or greater), diabetes, and overweight or obesity. On a population basis, a reduction of 2 mm Hg in diastolic blood pressure is estimated to result in a 15% reduction in risk of stroke and a 6% reduction in risk of coronary heart disease.

As used herein, an “effective amount” is an amount that prevents a deficiency, treats a disease or medical condition in an individual or, more generally, reduces symptoms, manages progression of the diseases or provides a nutritional, physiological, or medical benefit to the individual. The relative terms “improved,” “increased,” “enhanced” and the like refer to the effects of the composition comprising 4′-O-glucuronide epicatechin (disclosed herein) relative to a composition lacking 4′-O-glucuronide epicatechin but otherwise identical.

“Animal” includes, but is not limited to, mammals, which includes but is not limited to, rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the term “patient” is understood to include an animal, especially a mammal, and more especially a human that is receiving or intended to receive treatment, as treatment is herein defined. While the terms “individual” and “patient” are often used herein to refer to a human, the present disclosure is not so limited. Accordingly, the terms “individual” and “patient” refer to any animal, mammal or human that can benefit from the treatment. The animal, mammal or human may be a pregnant animal, mammal or human.

“Overweight” is defined for a human as a BMI between 25 and 30. “Obese” is defined for a human as a BMI greater than 30. “Weight loss” is a reduction of the total body weight. Weight loss may, for example, refer to the loss of total body mass in an effort to improve fitness, health, and/or appearance. “Weight management” or “weight maintenance” relates to maintaining a total body weight. For example, weight management may relate to maintaining a BMI in the area of 18.5-25 which is considered to be normal. The term “GDM” as used herein refers to any degree of impaired glucose tolerance that onsets or is first recognized during pregnancy. The term “preeclampsia” as used herein refers to a pregnancy condition diagnosed by high blood pressure and one or more of the following complications after the 20^{th} week of pregnancy: protein in urine, a low platelet count, impaired liver function, signs of kidney trouble other than protein in urine, fluid in the lungs (pulmonary edema), visual disturbances, new (onset) headaches. The term IUGR is a condition wherein the growth of an individual's foetus/baby is compromised and restricted in-utero so that said foetus/baby is smaller for gestational age. IUGR may result in said foetus/baby being a low birth weight baby.

As set forth above, the present inventors believe that 4′-O-glucuronide epicatechin can be used in methods that modulate nitric oxide (NO) levels. NO is important for relaxation of blood vessels and delivery of blood flow to tissues in the body. With improved blood flow, nutrients and other compounds in the blood can be delivered more efficiently to the skeletal muscle tissues. Furthermore, NO is an anabolic signal as well as a facilitator for stimulation of protein synthesis and release of growth factors such as polyamines. NO also leads to release of insulin and IGF-1, leading to increased uptake of anabolic substrates and also bio-utilization of the substrates. NO is also involved in immune function via T-cell stimulation. It has been suggested that aberrant NO synthesis and/or blood flow in the uterus and/or placenta may contribute to the pathophysiologic changes seen in preeclampsia and IUGR. Preeclampsia is a leading cause of maternal death throughout the world and is responsible for significant baby morbidity and mortality. Furthermore, preeclampsia has healthcare implications for the women later in life with those having suffered having an increased risk of hypertension, coronary artery disease, stroke and type 2 diabetes. IUGR is a major cause of perinatal and neonatal mortality and morbidity and is associated with an increased risk of several health problems in later life for the foetus/baby e.g. an increased risk of cardio vascular disease and type II diabetes.

Accordingly, an aspect of the present disclosure is a method for blood vessel dilation and/or increased delivery of blood flow to tissues in the body comprising administering a composition comprising 4′-O-glucuronide epicatechin to an individual. The composition can be administered to an individual at least once a day for at least one week, preferably for at least one month. The composition may be administered pre-pregnancy and in the case of a pregnant individual the composition can be administered for any period of pregnancy.

The composition can be administered to an individual having or at risk of high blood pressure and/or a cardiovascular disease and/or Preeclampsia and/or IUGR. In an embodiment, the composition is administered to the individual to provide an amount of the 4′-O-glucuronide epicatechin that achieves a therapeutic effect selected from the group consisting of reduced blood pressure, stimulation of protein synthesis, improvement of blood circulation, improvement of blood circulation in the brain, increased release of growth factors, enhanced immune function, and combinations thereof, the method comprising administering to the individual a composition comprising 4′-O-glucuronide epicatechin. In some embodiments, the increased blood flow from the 4′-O-glucuronide epicatechin can be used to treat or prevent erectile dysfunction. With respect to preeclampsia and/or IUGR, the composition can be administered for any period of pregnancy, or pre-pregnancy when a woman is trying to get pregnant. Since preeclampsia is most likely to occur in the 3^{rd} trimesters of pregnancy it may be beneficial if the composition is administered in the 3^{rd} trimester of pregnancy.

In an embodiment, the composition is administered to the individual to provide an amount of the 4′-O-glucuronide epicatechin that improves a characteristic selected from the group consisting of insulin resistance, glucose tolerance and a combination thereof. The composition can thereby reduce glycemia. In such embodiments, the individual can be an infant (a child under the age of 12 months) born preterm and/or experiencing intrauterine growth restriction (IUGR), a pregnant woman suffering from or at risk of suffering from gestational diabetes mellitus (GDM) or a lactating woman; or a child (up to twelve years of age), an adolescent (twelve to eighteen years of age), or an adult (over eighteen years of age) suffering from insulin resistance and/or type II diabetes, such as an animal such as a human In an embodiment, the method can comprise identifying the individual as in need of an improvement in insulin sensitivity and/or glucose tolerance. Gestational Diabetes Mellitus (GDM) is a condition specifically affecting pregnant women, it is a condition that can increase the risk of a number of maternal-fetal conditions, including macrosomia, birth injury, shoulder dystocia, premature delivery, and caesarian delivery. Mothers suffering from GDM also have an increased risk of developing type II diabetes immediately after pregnancy and later in life. Also, the foetus/infant of mothers suffering from GDM have an increased risk of developing an impaired glucose tolerance and/or suffering from excess weight/adiposity and associated metabolic disorders e.g. type II diabetes and obesity. With respect to GDM the composition can be administered for any period of pregnancy, or pre pregnancy when a woman is trying to get pregnant. Since GDM is most likely to occur in the 2^{nd} and 3^{rd} trimesters it may be beneficial if the composition is administered in the 2^{nd} and/or 3^{rd} trimester of pregnancy.

There is a direct link between glucose tolerance and mood, memory and cognition. For example, in a study in which participants were given an oral glucose tolerance test and cognitive tests, the older age group showed that those with poorer glucose tolerance forgot more words and had slower decision times; and, in those participants with poor glucose tolerance, a tendency for blood glucose to fall below baseline values was associated with better mood and faster working memory. Therefore, without being bound by theory, the inventors believe that 4′-O-glucuronide epicatechin can modulate blood flow to enhance insulin sensitivity and/or glucose tolerance, which in turn can improve one or more of mood, memory or cognition.

Accordingly, in an embodiment, the composition can be administered to provide an amount of 4′-O-glucuronide epicatechin that improves one or more of cognitive performance, cognition, mood or memory in an individual. The composition can treat or prevent one or more of cognitive decline, mild cognitive impairment, dementia, a mood disorder, or memory loss in an individual having or at risk of having one or more of these conditions. The method can comprise identifying the individual as having one or more of cognitive decline, mild cognitive impairment, dementia, a mood disorder, or memory loss, e.g., before initial administration of the composition. The method can comprise identifying the individual as being in need of an improvement in one or more of cognitive performance, cognition, mood or memory, e.g., before initial administration of the composition.

The composition can be administered to an infant (a child under the age of twelve months), a child (up to twelve years of age), an adolescent (twelve to eighteen years of age), an adult (over eighteen years of age), or an elderly individual (past the first two thirds of the average expected lifespan in its country of origin, preferably past the first three quarters of the average expected lifespan in its country of origin; an elderly human is a person with a chronological age of 65 years or older). The composition may be particularly effective against cognitive aging, and thus this embodiment of the method preferably comprises administration to an elderly human.

Cognitive performance may be expressed as ability and speed of learning, ability and speed of solving intellectual problems, ability to form and recall memories, reaction time, and the like. Cognition is understood as mental processes such as comprehension, inference, decision-making, planning, learning, memory, association, concept formation, language, attention, perception, action, problem solving and mental images. Cognitive decline may manifest as reduced memory; forgetfulness; word or name-finding problems; and/or decline in memory, concentration, ability to plan or organize, ability to perform complex tasks, and/or cognitive performance; and may result from age, stress, disease, or other grounds. Cognitive impairment may manifest in one or more of short-term memory loss, diminished capacity to learn, diminished rate of learning, or diminished attention.

The term “mood” refers to a state or quality of feeling (an emotional state) at a particular time. Moods differ from simple emotions in that they are less specific, less intense, and less likely to be triggered by a particular stimulus or event. Moods generally have either a positive or negative valence. An improved mood may comprise one or more of a decreased anxiety level, a decreased stress level, an increased perceived energy level, or a more positive emotional state.

In another aspect of the present disclosure, the composition comprising 4′-O-glucuronide epicatechin can be used in a method to support weight management or promote weight loss. For example, the composition can be administered to an individual, such as a mammal, that is managing their weight or undergoing a weight loss program. The weight loss program may include, for example, a weight loss diet (e.g., one or more of a low-fat diet, for example a diet with less than 20% of the calories from fat, preferably less than 15% from fat; a low-carbohydrate diet, for example a diet with less than 20% of the calories from carbohydrates; a low-calorie diet, for example a diet with less calories per day relative to the individual's previous intake before the diet, or a diet with less calories per day relative to an average person of similar body type; or a very low-calorie diet, for example a diet with 800 kcal (3,300 kJ) per day or less). Additionally or alternatively, the weight loss program may include a weight loss training regimen (e.g. endurance and/or strength training).

The individual can be overweight or obese or at risk thereof. The composition can also comprise an additional weight loss ingredient. The method can comprise identifying the individual as being in need of weight management or weight loss and/or identifying the individual as obese or overweight, e.g., before initial administration of the composition. The composition can be administered to provide an amount of the 4′-O-glucuronide epicatechin that increases at least one characteristic selected from the group consisting of energy expenditure, sympathetic nervous system activity, and fat oxidation.

In an embodiment, the composition comprising 4′-O-glucuronide epicatechin can be used in methods for treating or preventing endothelium dysfunction. For example, the composition comprising 4′-O-glucuronide epicatechin can be administered to an individual, such as a mammal, that has or is at risk of endothelium dysfunction to treat or prevent the endothelium dysfunction. The composition can be administered to the individual at least once a day for at least one week, preferably for at least one month.

The method can comprise identifying the individual as having endothelium dysfunction, e.g., before initial administration of the composition. The method can comprise identifying the individual as being at risk of endothelium dysfunction, e.g., before initial administration of the composition.

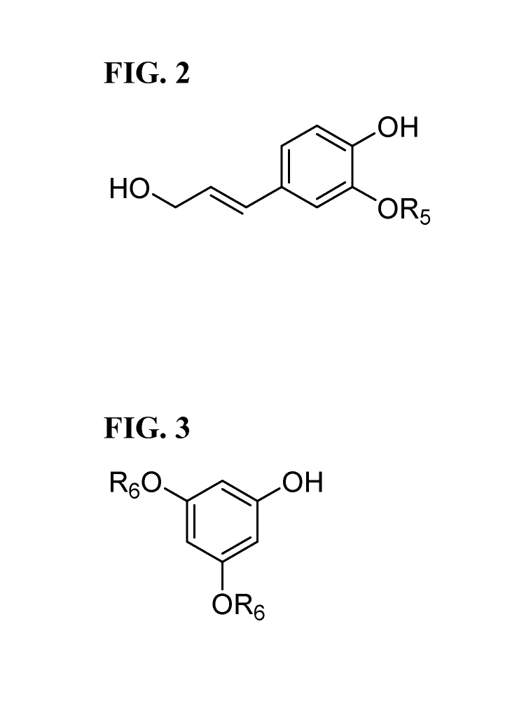

In any of the methods disclosed herein, the composition can comprise an extract enriched with 4′-O-glucuronide epicatechin. In an embodiment, the 4′-O-glucuronide epicatechin can be synthesized. For example, the 4′-O-glucuronide epicatechin can be synthesized as disclosed in WO2013/020979. As set forth therein, a first compound (FIG. 2) can be coupled to a second compound (FIG. 3) and then subjected to a stereo-selective intra-molecular trans-cyclization reaction to form 4′-O-glucuronide epicatechin. In the first compound, R5 is a protecting group. In the second compound, R6 is a protecting group.

The process can further comprise a Sharpless dihydroxylation reaction step after the coupling reaction and before the stereo-selective intra-molecular trans-cyclization reaction. This reaction allows introduction of chirality at C3 in the flavan-3-ol framework with high optical purity. The dihydroxylation can be performed according to Sharpless et al. (J. Org. Chem., 57(10): 2768-2771 (1992) and Chem. Rev. (Washington, D.C.), 94(8): 2483-2547 (1994)) with commercially available stereoselective catalysts AD-mix-α or AD-mix-β.

In an embodiment, the process further comprises an inversion of the configuration at position C3 of the cyclized product from the stereo-selective intra-molecular trans-cyclization reaction. This inversion can be based on a two-step oxidation/reduction sequence (Wan et al., Bioorg. Med. Chem., 12(13): 3521-27 (2004) and Viton et al., Eur. J. Org. Chem., 36:6069-78 (2008)). A Dess-Martin oxidation of the (R,S)-catechin derivative leads to the corresponding ketone compound with 2R absolute stereochemistry without deterioration of optical purity. Subsequent diastereoselective hydride reduction with L-Selectride® affords the corresponding (2R,3R)- epicatechin derivative while preserving the optical purity. Hence, the inversion can provide epicatechin conjugate molecules with high optical purity.

Preferably, the trans-cyclization reaction is performed in two sequential steps, first in the presence of triethylortho acetate and then followed by a deacetylation reaction at position C3 under reductive conditions. Cyclization using triethyl orthoformate under catalytic pyridinium p-toluenesulfonate conditions proceeds with high stereoselectivity and advantageously allows isolation of the target cyclized product in high purity in the form of a protected acetate ester. The resulting 3-acyl protected (R,S)-catechin derivative can then be de-acetylated under mild reductive conditions employing, e.g., DIBAL-H, to afford the desired enantiomer in high yields.

The protecting groups of the first and second compounds can be selected from the group consisting of allyl ether, benzyl ether, tert-butyl ether, tetrahydropyranyl ether, methoxymethyl ether, trimethylsilyl ether tert-butyldimethylsilyl (TBDMS), tert-butyldiphenylsilyl ether, trimethylsilyl ether, and tert-butyldimethylsilyl. Preferably, the protecting group is benzyl ether and/or TBDMS.

The process can further comprise a conjugation of the cyclized product from the stereo-selective intra-molecular trans-cyclization reaction after the inversion of the configuration, with a glucuronic acid donor. This reaction step allows generating the desired 4′-O-glucuronide epicatechin. For the above process step, the glucuronic acid donor is preferably selected from 2,3,4 -tri-O-acetyl-α-D-methylglucuronopyranosyl-1-(N-phenyl)-2,2 ,2 -trifluoroacetimidate; 2,3,4-tri-O-acetyl-α-D-methylglucopyranosyl-1-(N-4-methoxyphenyl)-2,2,2-trifluoroacetimid ate; 2,3,4-tri-O-acetyl-α-D-methylglucuronopyranosyl-1-O-(2,2,2 -trichloroacetimidate); or acetobromo-α-D-glucuronic acid methyl ester; and the glucose donor is preferably selected from 2,3,4,6 -tetra-O-acetyl-α-D-glucopyranosyl-1-(N-phenyl)-2,2,2 -trifluoroacetimidate; 2,3,4,6 -tetra-O-acetyl-α-D-glucuropyranosyl-1-O-(2,2,2 -trichloroacetimidate) or α-acetobromoglucose.

In an embodiment, the 4′-O-glucuronide epicatechin is synthesized using the process disclosed in “Epicatechin B-Ring Conjugates: First Enantioselective Synthesis and Evidence for Their Occurrence in Human Biological Fluids” by Romanov-Michailidis et al., Org. Lett., 14(15):3902-3905 (2012).

The 4′-O-glucuronide epicatechin can be co-administered with a polyphenol to increase the bioavailability of the 4′-O-glucuronide epicatechin, as disclosed in WO2014/083172. For example, the composition can comprise 4′-O-glucuronide epicatechin and at least one polyphenolic compound selected from the group consisting of flavonols, flavones, isoflavones, flavanones, and combinations thereof. In a preferred embodiment, a flavone and/or flavanone is selected from the group consisting of isorhamnetin, kaempfernol, diosmetin, nevadensin, chrysin, hesperitin, and combinations thereof.

The composition comprising 4′-O-glucuronide epicatechin may be a medicament, a food product, a medical food, an oral nutritional supplement, a nutritional composition, an oral cosmetic, or a supplement to a food product and is preferably orally administered. A medical food product is specially formulated and intended for the dietary management of diseases or medical conditions (e.g., prevent or treat diseases or undesirable medical conditions). A medical food product can provide clinical nutrition, for example fulfilling special nutritional needs of patients with a medical condition or other persons with specific nutritional needs. A medical food product can be in the form of a complete meal, part of a meal, as a food additive, or a powder for dissolution.

In an embodiment, the composition is administered orally as a food product in which the 4′-O-glucuronide epicatechin is present in a concentration of at least 0.01 mg/g of the food product, preferably at least 0.1 mg/g of the food product, more preferably at least 1 mg/g of the food product, even more preferably 10 mg/g of the food product, or more.

A food product, medical food or nutritional composition includes any number of optional additional ingredients, including conventional food additives, for example one or more proteins, carbohydrates, fats, acidulants, thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers and/or vitamins. The optional ingredients can be added in any suitable amount.

A food product, medical food or nutritional composition can be in any oral nutritional form, e.g. as a health drink, as a ready-made drink, optionally as a soft drink, including juices, milk-shake, yogurt drink, smoothie or soy-based drink, in a bar, or dispersed in foods of any sort, such as baked products, cereal bars, dairy bars, snack-foods, soups, breakfast cereals, muesli, candies, tabs, cookies, biscuits, crackers (such as a rice crackers), and dairy products.

A supplement may be in the form of tablets, capsules, pastilles or a liquid, for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins or the like), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like. The supplement may for example be a maternal supplement for administration to an individual pre pregnancy when said individual is trying to get pregnant, and/or during pregnancy and/or during lactation.

The supplement can be added in a product acceptable to the consumer as an ingestible carrier or support. Non-limiting examples of such carriers or supports are a pharmaceutical, a food composition, and a pet food composition. Non-limiting examples for food and pet food compositions are milks, yogurts, curds, cheeses, fermented milks, milk-based fermented products, fermented cereal based products, milk-based powders, human milks, preterm formulas, infant formulas, oral supplements, and tube feedings.

In some embodiments, the composition comprising 4′-O-glucuronide epicatechin is in an injectable form, and the methods disclosed herein can comprise injecting the individual with the composition.

### EXAMPLE

Comparative experiments were conducted on the expression of endothelial nitric oxide synthase in isolated aortic rat rings.

Thoracic aortas were obtained from male adults Wistar rats after euthanasia. Each aorta was carefully dissected and harvested cleaned of excess tissue and cut into three equal segments, approximately 7 mm long. All aortic rings were pre-incubated for 30 min in Krebs solution (20 mM Hepes buffer, pH 7.4, containing 119 mM NaCl, 4.7 mM KCl, 1 mM MgSO₄, 0.4 mM NaH₂PO4, 0.15 mM Na₂HPO4, 5 mM NaHCO3, 1.25 mM CaCl_{2, 5.5} mM glucose) aerated with 95% O₂ and 5% CO₂ at 37° C. and then incubated with vehicle (DMSO 1%) or the different metabolites for 1 h. Metabolites were first dissolved in DMSO 1% and tested in a final concentration of 10 mM. At the end of the incubation period aortic rings were frozen at −70° C. until the homogenate preparation. Homogenates for western blot and western blot determinations were carried out by the regular procedures and phosphorylation level of endothelial nitric oxide synthase was evaluated.

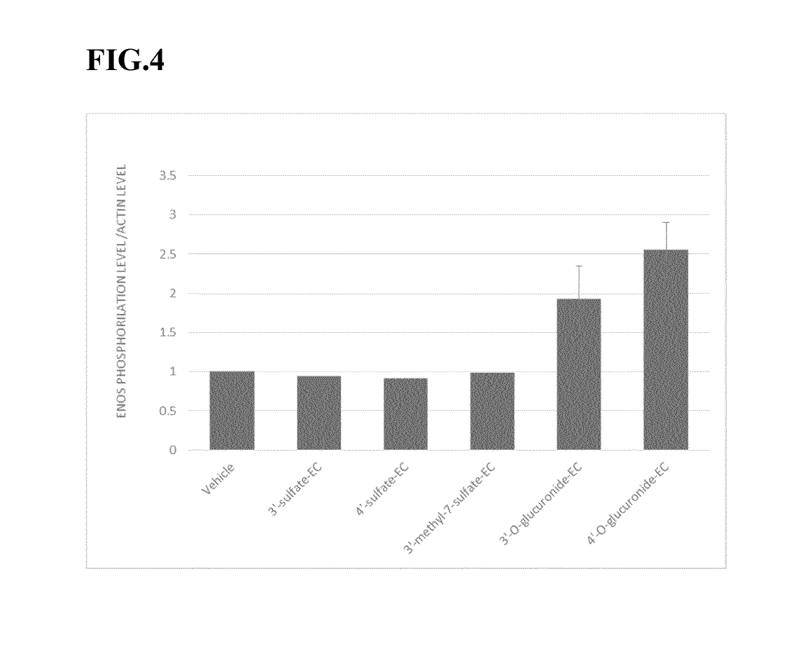

The results depicted in FIG. 4 demonstrate that 4′-O-glucuronide epicatechins increased eNOS phosphorylation relative to control and chemically similar epicatechins.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for blood vessel dilation and/or increased delivery of blood flow to tissues in the body, the method comprising administering a composition comprising 4′-O-glucuronide epicatechin to an individual.

2. The method of claim 1 wherein the composition is orally administered to the individual as a food product in which the 4′-O-glucuronide epicatechin is present in a concentration of at least 0.01 mg/g of the food product.

3. The method of claim 1 wherein the composition is administered to the individual at least once a day for at least one week.

4. The method of claim 1 wherein the 4′-O-glucuronide epicatechin is chemically synthesized.

5. (canceled)

6. The method of claim 1 wherein the individual has or is at risk of a cardiovascular disease.

7. The method of claim 1 wherein the composition is administered to the individual in an amount that achieves a therapeutic effect selected from the group consisting of reduced blood pressure, stimulation of protein synthesis, improvement of blood circulation, improvement of blood brain circulation, increased release of growth factors, enhanced immune function, and combinations thereof, the method comprising administering to the individual a composition comprising 4′-O-glucuronide epicatechin.

8. The method of claim 1 wherein the composition is administered to the individual in an amount that improves a characteristic selected from the group consisting of insulin resistance, glucose tolerance and a combination thereof.

9. The method of claim 8 wherein the individual is selected from the group consisting of an infant born preterm, an infant experiencing intrauterine growth restriction, a pregnant woman suffering from gestational diabetes, a human suffering from insulin resistance, a human suffering from impaired glucose tolerance, and a human suffering from type II diabetes.

10. The method of claim 1 wherein the composition is administered to the individual in an amount that improves one or more of cognitive performance, cognition, mood, or memory.

11. (canceled)

12. The method of claim 10 wherein the individual is an elderly human having cognitive aging.

13. The method of claim 1 wherein the composition is injected into the individual.

14. A method for weight maintenance or weight loss comprising administering a composition comprising 4′-O-glucuronide epicatechin to an individual.

15. The method of claim 14 wherein the composition is orally administered to the individual as a food product in which the 4′-O-glucuronide epicatechin is present in a concentration of at least 0.01 mg/g of the food product.

16. The method of claim 14 wherein the composition is administered to the individual at least once a day for at least one week.

17. The method of claim 14 wherein the 4′-O-glucuronide epicatechin is chemically synthesized.

18. The method of claim 14 wherein the composition further comprises an additional ingredient in an amount effective to promote weight maintenance or weight loss.

19. The method of claim 14 wherein the composition is administered to provide an amount of the 4′-O-glucuronide epicatechin that increases at least one characteristic selected from the group consisting of energy expenditure, sympathetic nervous system activity, and fat oxidation.

20. (canceled)

21. The method of claim 14 wherein the individual is participating in a weight loss program selected from the group consisting of a low-fat diet, a low-carbohydrate diet, a low-calorie diet, a very low-calorie diet, endurance training, strength training, and combinations thereof.

22. (canceled)

23. A method for treating or preventing a condition selected from the group consisting of endothelium dysfunction, preeclampsia and/or IUGR, and gestational diabetes mellitus comprising administering to an individual in need thereof or at risk thereof a composition comprising 4′-O-glucuronide epicatechin.

24. The method of claim 23 wherein the composition is orally administered to the individual as a food product in which the 4′-O-glucuronide epicatechin is present in a concentration of at least 0.01 mg/g of the food product.

25. The method of claim 23 wherein the composition is administered to the individual at least once a day for at least one week.

26. (canceled)

27. The method of claim 23 wherein the composition is orally administered to the individual as a maternal supplement in which the 4′-O-glucuronide epicatechin is present in a concentration of at least 0.01 mg/g of the composition.

28. The method of claim 23 wherein the composition is administered to the individual pre-pregnancy when said individual is trying to get pregnant, and/or during pregnancy.

29-30. (canceled)

31. The method of claim 23 wherein the composition is administered to the individual pre-pregnancy when said individual is trying to get pregnant and/or, during pregnancy, and or during lactation.

32. The method according to claim 23 wherein the composition is in the form of an extract enriched with 4′-O-glucuronide epicatechin.

33. (canceled)
